(19) United States
(12) Patent Application Publication
(10) Pub. No.: US 2012/0226210 A1
(43) Pub. Date: Sep. 6, 2012

(54) CONFIGURABLE FOOT ORTHOSIS

(75) Inventors: Jean-François Normandin, Carignan (CA); Sylvain Guimond, Sainte-Anne-de-Sorel (CA)

(73) Assignee: BIOTONIX (2010) INC., Brossard, QC (CA)

(21) Appl. No.: 13/383,715

(22) PCT Filed: Jul. 7, 2010
(86) PCT No.: PCT/CA10/01078

§ 371 Date: May 22, 2012

Related U.S. Application Data
(60) Provisional application No. 61/213,766, filed on Jul. 13, 2009.

(51) Int. Cl. A61F 5/00

(52) U.S. Cl. 602/23

(57) ABSTRACT

There is disclosed a configurable foot orthosis, comprising a corrective layer and a plurality of position adjusters, each position adjuster being associated with a respective area of the human foot. Each position adjuster can be adjusted in order to counteract a postural deviation.

### CROSS-REFERENCE TO RELATED APPLICATIONS

This application claims the benefits of U.S. provisional patent application No. 61/213,766 filed on Jul. 13, 2009, which is herein incorporated by reference.

### TECHNICAL FIELD

The present invention relates to a configurable foot orthosis.

### BACKGROUND

Posture is defined as the position of the limbs or the carriage of the body as a whole. Science has shown that good posture allows the body to function and perform optimally. On the other hand, bad posture causes muscular imbalances and pressures on the nerves and internal organs which can lead to musculoskeletal disorders, aches and pains, joint degeneration, and poor bodily functions. Since the feet are the base on which a person's weight rests, any inadequate orientation of the feet in the natural stance position requires compensation by the trunk and limbs, thereby inducing postural deviations.

Foot orthotics are frequently used to relieve the aches resulting from such compensations, or even from medical conditions such as lower limb length discrepancy, etc. These orthotics are labour intensive to produce and may require the intervention of a health specialist. They also require several days to complete. Moreover, the associated costs can render such technologies prohibitive when the main objective is posture improvement instead of the treatment of a medical condition. Self adjusted off-the-shelf insoles are available but they focus on comfort alone and any postural improvement is purely fortuitous. In some cases, they may be more harmful than beneficial as they are adjusted on a lay person's perception rather than a trained specialist or an objective analysis.

Accordingly, there is a need for a solution that provides a rapid production system for customized footwear based on a postural evaluation without prior anatomic knowledge or expertise from the system manipulator.

### SUMMARY

According to one aspect of the present invention, there is provided a configurable foot orthosis, comprising:
a corrective layer; and
a plurality of position adjusters, each position adjuster being associated with a respective area of the human foot;
   wherein each position adjuster can be adjusted in order to counteract a postural deviation.

According to another aspect of the present invention, there is provided a postural adjustment system, comprising:
a configurable foot orthosis as described above;
an imaging unit; and
a processing unit operatively connected to the configurable foot orthosis and the imaging unit, the processing unit being so configured so as to:
   acquire at least one digital image of an individual from the imaging unit;
   process the at least one digital image;
   identify one or more postural deviations of the individual;
   generate adjustments variables for the plurality of position adjusters in accordance with the one or more postural deviations;
   providing commands to adjust the plurality of position adjusters in accordance with the adjustment variables in order to correct the one or more postural deviations of the individual.

According to a further aspect of the present invention, there is provided a pressure point matrix for providing the necessary measurements to prepare a human foot imprint, comprising:
a body supporting a plurality of gauges, each gauge having an associated actuator and an associated sensor, the body including a top surface having a plurality of perforations therethrough, each perforation being configured so as to allow a corresponding gauge to protrude from the body;
   wherein when an individual steps onto the top surface, each sensor notes the position of its respective gauge pin, providing an imprint of the sole of the individual.

### BRIEF DESCRIPTION OF THE FIGURES

Embodiments of the invention will be described by way of examples only with reference to the accompanying drawings, in which:

FIG. 1 is a transparent exploded perspective view of the left foot of an illustrative embodiment of the configurable foot orthosis;

FIG. 2 is a schematic elevation view of a left foot positioned on the configurable foot orthoses of FIG. 1;

FIG. 3 is a schematic top view of the pair of configurable foot orthoses showing the positioning of distendable cavities;

FIG. 4 is a schematic view of the pair of configurable foot orthoses of FIG. 3 used with a postural diagnostic system;

FIG. 5 is a flow diagram of an example of a process showing the use of the pair of configurable foot orthoses and postural diagnostic system of FIG. 4;

FIG. 6 is a flow diagram of a postural analysis and adjustment variables generation sub-process used by the process of FIG. 5;

FIG. 7 is a schematic elevation view of a left foot positioned on a pressure point matrix;

FIG. 8 is a schematic top view of the pressure point matrix of FIG. 7; and

FIG. 9 is a schematic view of the pressure point matrix of FIG. 8 used with the pair of configurable foot orthoses and postural diagnostic system of FIG. 4.

### DETAILED DESCRIPTION

A non-restrictive illustrative embodiment of the present invention relates to a configurable foot orthosis, which helps correct postural deviations of an individual in a passive way. It is to be understood that the aim of the configurable foot orthosis is to correct the posture of the wearer, not only the geometry of the foot.

The configurable foot orthosis may be removable, to be used with any type of footwear, or built into the footwear itself. Examples of footwear in which the configurable foot orthosis may be integrated into include skates, ski boots, running shoes, hiking shoes/boots, work boots, etc.

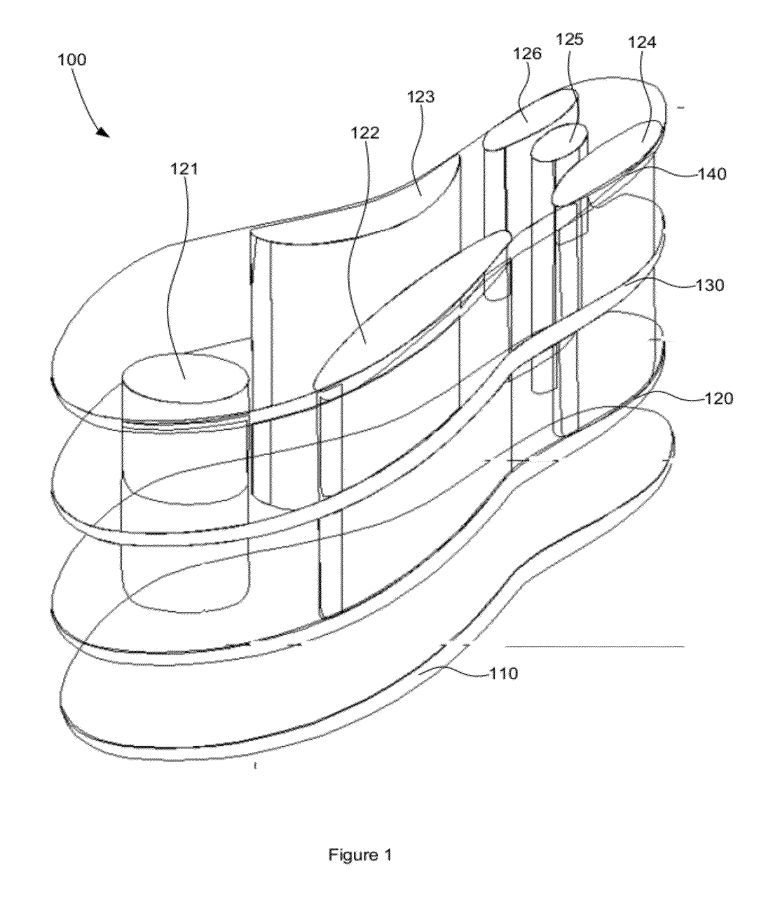

Referring to FIG. 1, the illustrative embodiment of the configurable foot orthosis 100 comprises four superimposed layers: a base layer or sole 110, a corrective layer 120, an imprint layer 130 and a cover layer or skin 140. The corrective layer 120 includes six position adjusters 121 to 126 such as, for example, cavities, placed at strategic areas of the foot. It is to be understood that the number position adjusters may vary, i.e. there may be less or more than six, and that their positioning may correspond to other strategic areas of the foot.

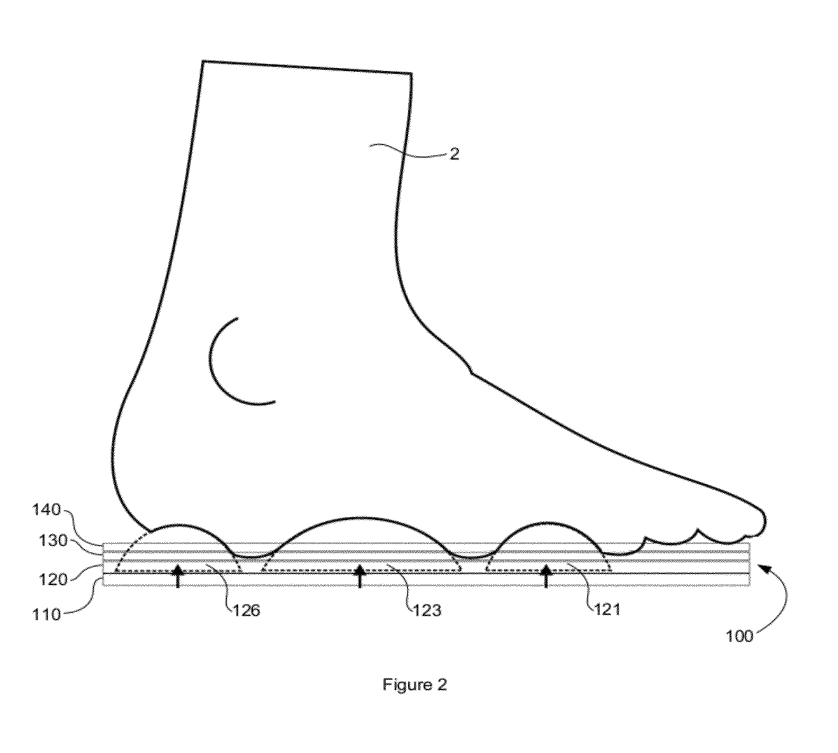

In use, as schematically shown in FIG. 2, the layers 110, 120, 130 and 140 of the configurable foot orthosis 100 are compressed together by the foot 2 of a user, the position adjusters 121, 122 (not visible), 123, 124 (not visible), 125 (not visible) and 126 applying pressure against their associated underfoot areas of the foot 2 through the imprint 130 and cover 140 layers in order to apply a desired postural correction.

It is to be understood that the configurable foot orthoses 100 can be provided in a plurality of sizes. It is also to be understood that the configurable foot orthosis 100 comprises at the minimum the corrective layer 120, that one or more of the other layers 110, 130 and 140 may be omitted and that additional layers may be added.

### Base Layer

The base layer 110 provides support for the configurable foot orthosis 100 and may be made, for example, of flexible thermoplastic such as polyethylene or any other appropriate material.

### Corrective Layer

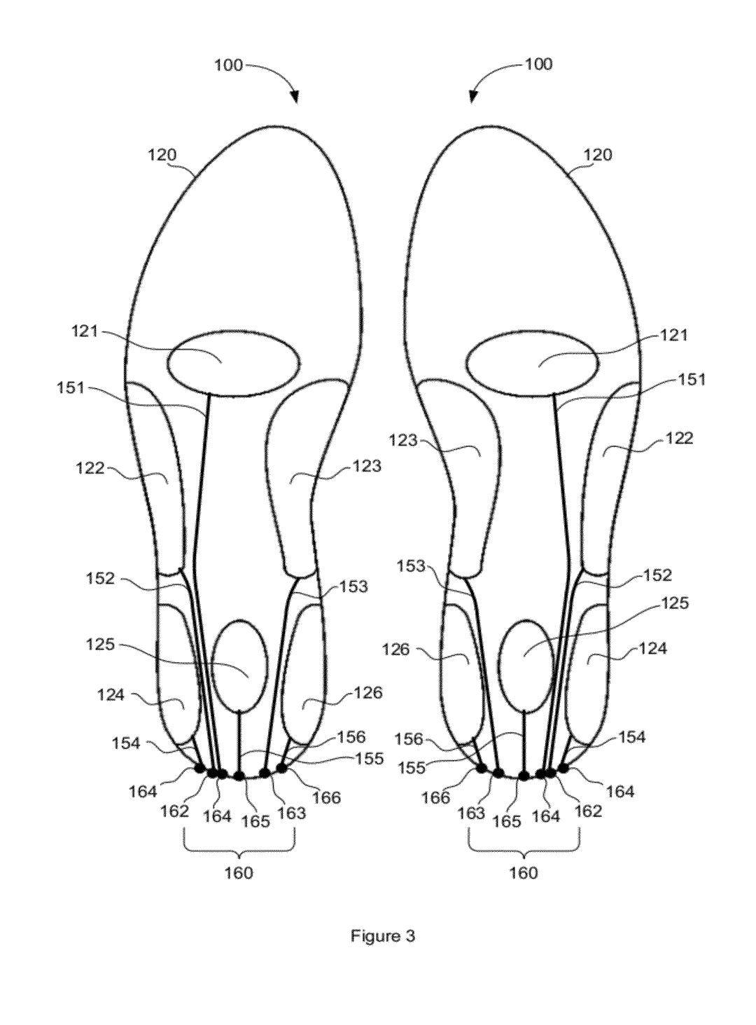

Referring to FIG. 3, the corrective layer 120 includes six position adjusters 121 to 126 associated with, respectively, the following areas of the foot: forefoot (121), lateral midfoot (122), medial midfoot (123), lateral hindfoot (124), central hindfoot (125) and medial hindfoot (126); in both a right and left foot configurations. As mentioned previously, the position adjusters 121 to 126 may be in the form of, for example, distendable cavities that may be filled with a gel, a liquid or a gas. In the present illustrative embodiment, the position adjusters 121 to 126 are in the form of distendable cavities, each of which having an associated cavity conduit 151 to 156 that may be accessed via an access interface 160. The corrective layer 120 and position adjusters 121 to 126 distendable cavities may be made, for example, of polyvinyl chloride (PVC), chlorosulfonated-polyethylene (CSPE/CSM) or any other appropriate material.

The access interface 160 comprises an access port 161 to 166 associated with, respectively, each cavity conduit 151 to 156. The access ports 161 to 166 and conduits 151 to 156 are used to fill their associated distendable cavities (i.e. position adjusters 121 to 126) to a desired volume so as to correct postural deviations. As previously mentioned, the distendable cavities 121 to 126 may be filled with a gel, a liquid or a gas, and their associated access ports 161 to 166 sealed once the distendable cavities 121 to 126 are filled to the desired volume.

Alternatively, a curable resin/gel may be used and cured once the desired volume is attained via, for example, a catalyser, heat, x-ray, etc., in which case sealing of the access ports 161 to 166 is not required.

In another alternative embodiment, the position adjusters 121 to 126 may be in the form of receiving cavities configured to receive support pads of various predefined volumes. The support pads may be made of, for example, ethyl-vinyl acetate (EVA) or polyurethane foam blocs. It is to be understood that access to the receiving cavities is provided, for example, by having removable or partially removable imprint 130 and cover 140 layers.

In a further embodiment, the position adjusters 121 to 126 may be in the form of actuators, for example set screws, which may be manually adjusted in order to vary their respective height.

### Imprint Layer

The imprint layer 130 allows for the comfort of the individual by conforming to the sole of his or her foot and may be made, for example, of a polyvinyl chloride (PVC), chlorosulfonated-polyethylene (CSPE/CSM) or any other appropriate material, and contain a thermosetting resins/gel. The imprint layer 130 may be formed by having the individual step onto the imprint layer 130 and then thermosetting the resins/gel within. Other techniques that may be used include scanning of the sole of the feet of the individual, using a pressure point matrix, etc.

### Cover Layer

The cover layer 140 protects the configurable foot orthosis 100 from wear and tear, and may be made of, for example, leather, hypo allergenic expended thermoplastic such as EVA, polyurethane foam, etc. The cover layer 140 may be an integral part of the configurable foot orthosis 100 or may be removable in order to allow its easy replacement.

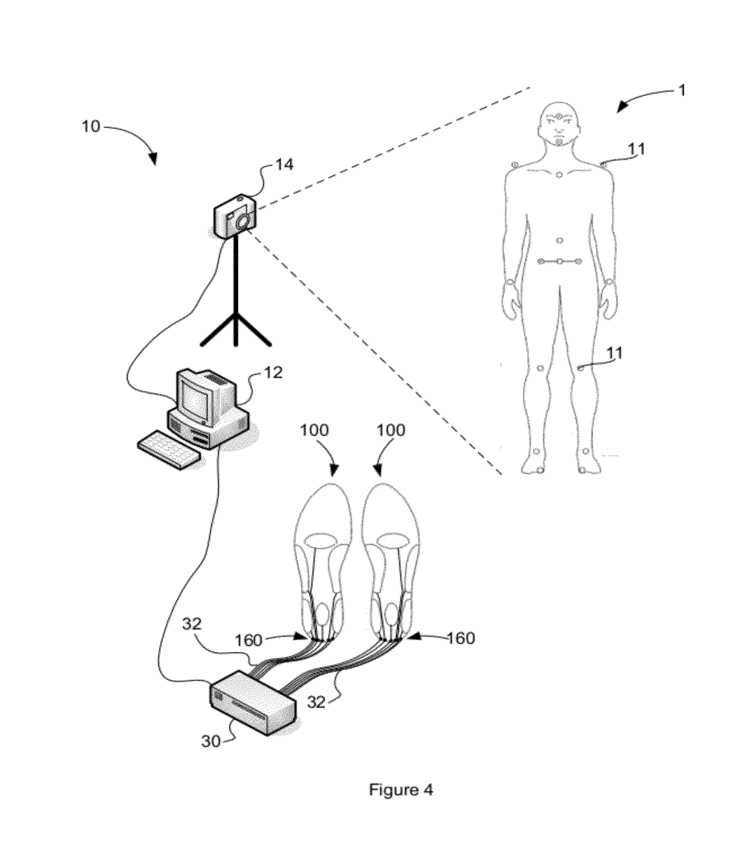

Referring to FIG. 4, the configurable foot orthosis 100 may be used with a postural diagnostic system 10 in order to provide a postural adjustment system. The postural diagnostic system 10 generally comprises a processing unit 12, such as a computer, and an imaging unit 14, such as a digital camera or scanner. One or more digital images of an individual 1 acquired by the imagining unit 14 are treated by the processing unit 12 in order to establish a postural diagnostic of the individual 1. The processing unit 12 may use various techniques such as the analysis of the relative position of markers 11 positioned at specific locations on the individual 1. An example of a postural diagnostic system 10 that may be used is described in U.S. Pat. No. 6,514,219 entitled “SYSTEM AND METHOD FOR AUTOMATED BIOMECHANICAL ANALYSIS AND THE DETECTION AND CORRECTION OF POSTURAL DEVIATION” by Guimond et al.

A configuration interface 30 implements a correspondence table which, used together with other related data from the processing unit 12, determines the proper adjustments to be made for one or more of the position adjusters 121 to 126 in order to correct the posture of the individual 1. The configuration interface 30 is connected to the access interfaces 160 of a pair configurable foot orthoses 100 (or only a single orthosis) via configuration conduits 32 so as to automatically configure the configurable foot orthosis 100 in accordance with the diagnosis data obtained from the processing unit 12. This is achieved by filling the distendable cavities (i.e. position adjusters 121 to 126, which are best seen in FIG. 3) to the desired volume so as to correct the postural deviations. As previously mentioned, the distendable cavities 121 to 126 may be filled by the configuration interface 30, which may comprise an injection mechanism, with a gel, a liquid or a gas, and the associated access ports 161 to 166 sealed once the distendable cavities 121 to 126 are filled to the desired volume. Alternatively, algorithms or computations may be substituted for the correspondence table.

It is to be understood that the postural diagnostic system 10 may be integrated into a kiosk allowing onsite preparation of one or more configurable foot orthosis 100 while the individual 1 waits.

In an alternative embodiment, the correspondence table and other related data may be implemented within the processing unit 12 so as to directly provide the proper adjustments to be made for one or more of the position adjusters 121 to 126 to the configuration interface 30.

In a further alternative embodiment, the processing unit 12 may provide the proper adjustments to be made for one or more of the position adjusters 121 to 126 in order for a professional to manually implement the generated adjustments, either via a mechanism embedded within the configurable foot orthosis 100 or a further apparatus.

Table 1 is an example of a correspondence table identifying the position adjusters that are to be adjusted in order to counteract postural deviations identified by the postural diagnostic system 10. It is to be understood that the specific adjustments depend on the severity of the diagnosed postural deviation.

TABLE 1
Correspondence between postural deviations and
position adjusters

| Deviation | Position Adjuster(s) |
|---|---|
| Head Forward Protrusion | 123 (right and left) |
| Right Lateral Neck Flexion | 123 (right) |
| Left Lateral Neck Flexion | 123 (left) |
| Right Shoulder Elevation | 123 (right) |
| Left Shoulder Elevation | 123 (left) |
| Shoulder Protraction/Kyphosis | 123 (right and left) |
| Trunk Positive Rotation | 124 (left) and/or 126 (right) |
| Trunk Negative Rotation | 124 (right) and/or 126 (left) |
| Right Pelvis Elevation | 125 (left) |
| Left Pelvis Elevation | 125 (right) |
| Forward Pelvic Tilt | 123 (right and left) |
| Backward Pelvic Tilt | 122 (left and right) |
| Pelvic Positive Rotation Y axis | 124 (left) and/or 126 (right) |
| Pelvic Negative Rotation Y axis | 124 (right) and/or 126 (left) |
| Pelvic Positive Translation X axis | 122 (left) and/or 123 (right) |
| Pelvic Negative translation X axis | 122 (right) and/or 123 (left) |
| Pelvic Positive Translation Z axis | 121 (right and left) |
| Pelvic Negative Translation Z axis | 125 (right and left) |
| Genu Valgum | 123 (right and left) |
| Genu Varum | 122 (right and left) |
| Foot Pronation | 123 (right and left) |
| Foot Supination | 122 (right and left) |
| External Foot Rotation | 124 (right and left) |
| Internal Foot Rotation | 126 (right and left) |

It is to be understood that the correspondence table may contain other related data such as, for example, the desired amplitude of the adjustment depending on the severity of the postural deviation as well as any considerations for multiple simultaneous postural deviations, historic of the individual, etc.

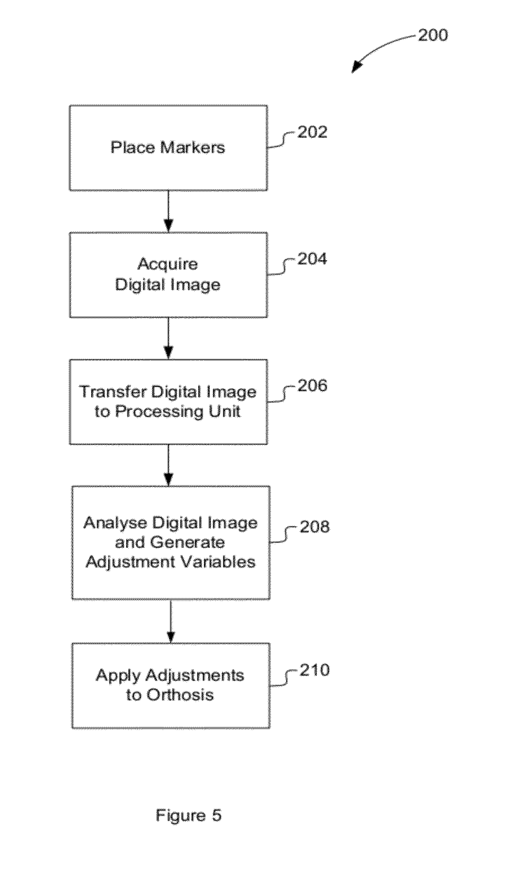

Referring now to FIG. 5, there is shown a flow diagram of an example of a process showing the use of the configurable foot orthosis 100 with a postural diagnostic system 10 such as the one described in U.S. Pat. No. 6,514,219. The steps of the process 200 are indicated by blocks 202 to 210, with references to FIG. 4.

The process 200 starts at block 202 where position markers 11 are positioned on the body of the individual 1 at specific anatomical locations.

At block 204, one or more digital images of the individual 1 are acquired by the imagining unit 14 and provided, at block 206, to the processing unit 12.

At block 208, the processing unit 12 executes a postural analysis and adjustment variables generation sub-process on the data of the one or more digital images in order to establish a postural diagnostic of the individual 1 and generate adjustment variables for the position adjusters 121 to 126 in order to treat one or more diagnosed postural deviations.

Then, at block 210, the position adjusters 121 to 126 are adjusted according to adjustment variables generated at block 208. Depending on the specifics of the postural diagnostic system 10, the position adjusters 121 to 126 may be adjusted automatically via a configuration interface 30, the correspondence table and other related data being either implemented in the processing unit 12 or the configuration interface 30. In an alternative embodiment, where support pads are placed within receiving cavities corresponding to the position adjusters 121 to 126 instead of injection a gel, liquid or gas, appropriate sizes support pads may be inserted within the receiving cavities or actuators adjusted in accordance with the generated adjustment variables.

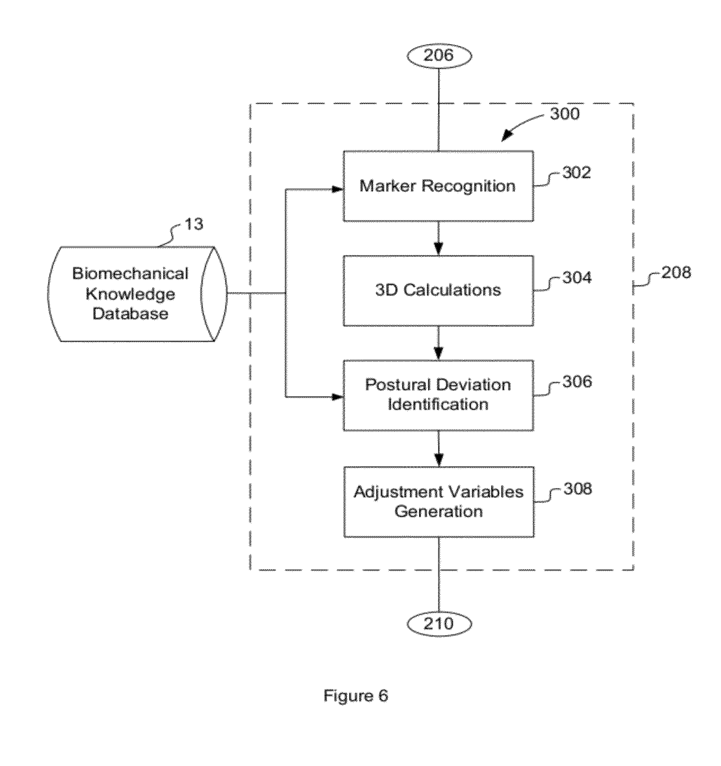

Referring to FIG. 6, there is shown a flow diagram of an example of a postural analysis and adjustment variables generation sub-process 300 used at block 208 of process 200 (see FIG. 5). The steps of the sub-process 300 are indicated by blocks 302 to 308.

The sub-process 300 starts at block 302 where the position markers 11 are identified in the one or more digital images of the individual 1 using data from a biomechanical knowledge database 13 and their three-dimensional (3D) coordinates in space are determined. The biomechanical knowledge database 13 may be incorporated into the processing unit 12, provided as a separate database in the postural diagnostic system 10 or accessed remotely from a further system and/or database.

At block 304, various deviations and biomedical parameters are computed by comparing the 3D coordinates of the position markers 11 determined at block 302 with corresponding expected 3D coordinates in a healthy individual.

Then, at block 306, using the computed deviations and biomedical parameters, postural deviations are identified using the biomechanical knowledge database 13.

Finally, at block 308, using the identified postural deviations, adjustment variables for the position adjusters 121 to 126 are generated in order to correct the deviations. These adjustment variables may be generated, as previously mentioned, by the processing unit 12 or the configuration interface 30.

### Pressure Point Matrix

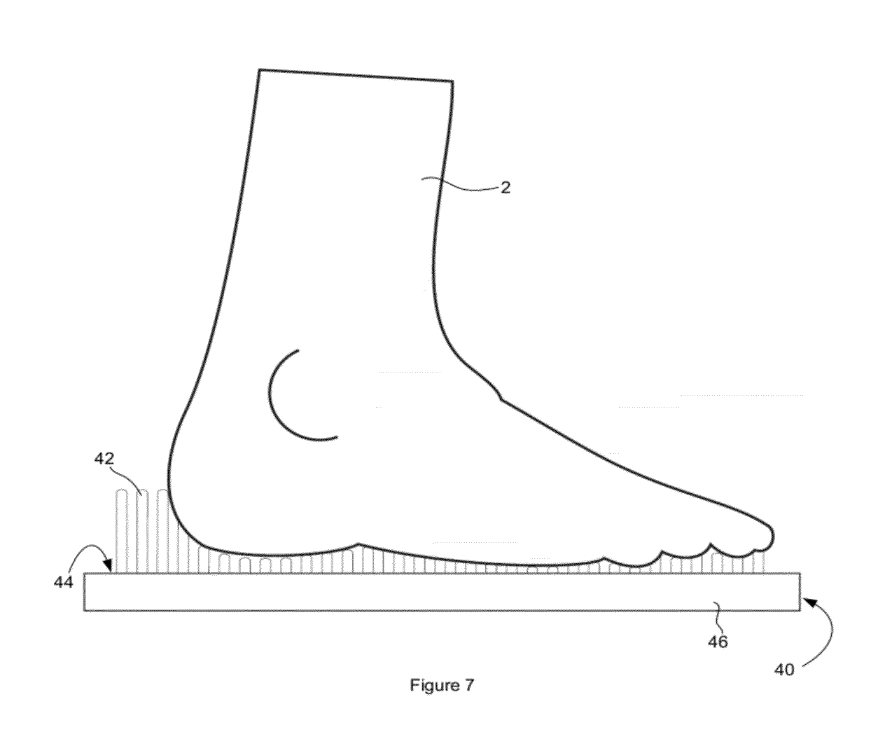

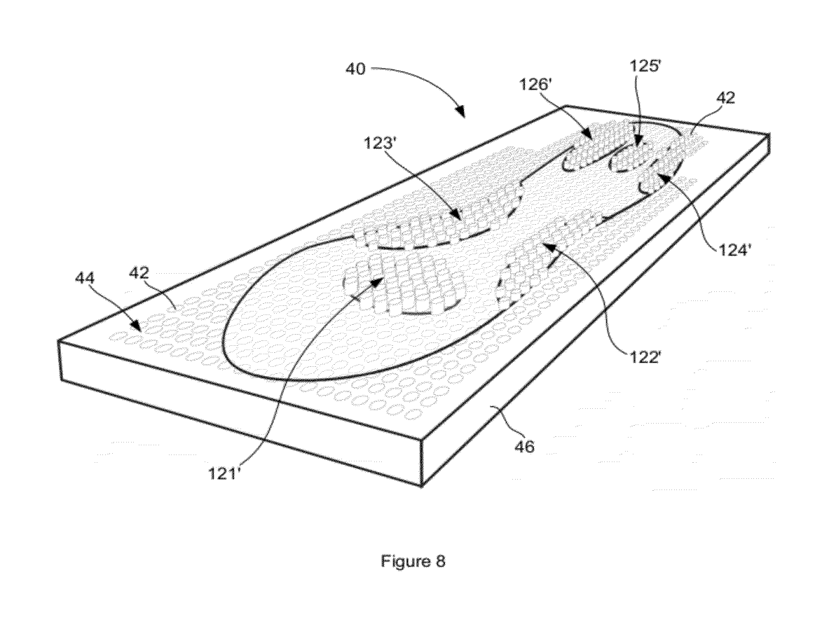

As previously mentioned, a pressure point matrix 40, for example the one illustrated in FIGS. 7 and 8, may be used in order to provide the necessary measurements for the preparation of the imprint layer 130. The pressure point matrix 40 comprises a plurality of gauge pins 42 that are pushed through a top perforated matrix 44 by actuators (not shown) located in the body 46 of the pressure point matrix 40. Each actuator is activated by, for example, compressed air, hydraulic fluid or any other suitable mechanism, and has an associated sensor (not shown) that notes the gauge pin's 42 position, which serves as an imprint of the sole of the individual's foot 2.

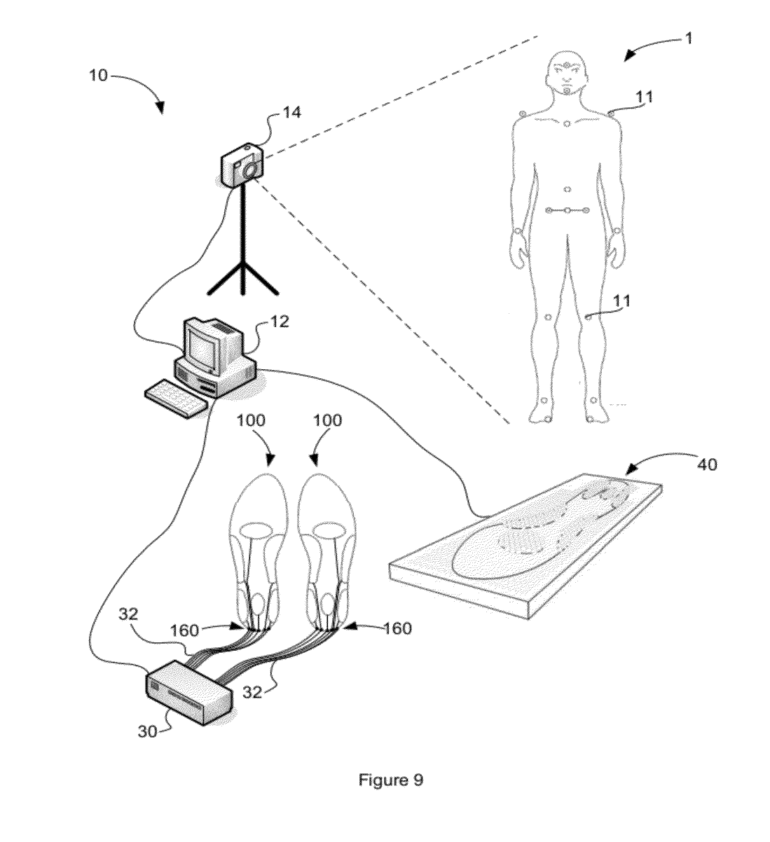

Referring to FIG. 9, the pressure point matrix 40 may be used with the configurable foot orthosis 100 and postural diagnostic system 10 in order to “fine tune” the adjustments provided by the processing unit 12 to the one or more position adjusters 121 to 126. After the individual's 1 postural deviations are determined by the postural diagnostic system 10, appropriate adjustments to one or more of the position adjusters 121 to 126 are first sent to the pressure point matrix 40 that activates the actuators of sets 121′ to 126′ of gauge pins 42 associated with respective position adjuster 121 to 126 (see FIG. 8).

The effect of the adjustments to the sets 121′ to 126′ of gauge pins 42 can then be viewed on the postural diagnostic system 10 by having the individual 1 step onto the pressure point matrix 40. Fine tuning of the adjustments to the one or more position adjusters 121 to 126 may then be made by the practitioner to in order optimize the correction, if necessary. The postural diagnostic system 10 then translates the tuned gauge pins 42 elevated positions, for each set 121′ to 126′, into a desired volume (CC) that can be injected in the corresponding distendable cavities or a desired height (mm) for the selection of the appropriate support pads or the adjustment of appropriate actuators. The optimized volumes or height are then provided to the corresponding position adjusters 121 to 126 of the configurable foot orthosis 100.

It is to be understood that the concept of the use of a corrective layer 120 may be applied to other applications, the number, position and size of the position adjusters varying depending on the application. For example, the corrective layer 120 may be in the form of a mattress including one or more position adjusters, for example distendable cavities that may be filled with a gel, a liquid or a gas, receiving cavities configured to receive support pads of various predefined volumes, actuators, etc. Such a mattress may be used, for example, as a configurable back orthosis on which an individual may lay in order to correct some postural deviations.

Although the present invention has been described by way of particular embodiments and examples thereof, it should be noted that it will be apparent to persons skilled in the art that modifications may be applied to the present particular embodiments without departing from the scope of the present invention.

1. A configurable foot orthosis, comprising:
a corrective layer; and
a plurality of position adjusters, each position adjuster being associated with a respective area of the human foot;
wherein each position adjuster can be adjusted in order to counteract a postural deviation.

2. The configurable foot orthosis of claim 1, comprising six position adjusters.

3. The configurable foot orthosis of claim 2, wherein the six position adjusters are associated with the forefoot, lateral midfoot, medial midfoot, lateral hindfoot, central hindfoot and medial hindfoot areas of the human foot.

4. The configurable foot orthosis of claim 1, wherein the position adjusters are distendable cavities.

5. The configurable foot orthosis of claim 4, further comprising a plurality of cavity conduit, a cavity conduit being associated with each distendable cavities, wherein the cavity conduits allow for the filling of the distendable cavities.

6. The configurable foot orthosis of claim 5, wherein the distendable cavities are filled with a material selected from the group consisting of a gel, a liquid, a gas a thermosetting resin and a thermosetting gel.

7. The configurable foot orthosis of claim 1, wherein the position adjusters are receiving cavities for receiving therein support elements.

8. The configurable foot orthosis of claim 1, wherein the position adjusters are actuators that can be adjusted in height.

9. The configurable foot orthosis of claim 1, further comprising a base layer, the corrective layer being positioned over the base layer, wherein the base layer provides support to the configurable foot orthosis.

10. The configurable foot orthosis of claim 9, wherein the base layer is made of a flexible thermoplastic material.

11. The configurable foot orthosis of claim 1, further comprising an imprint layer, being positioned over the corrective layer, wherein the imprint layer conforms to the foot of an individual using the configurable foot orthosis.

12. The configurable foot orthosis of claim 11, wherein the imprint layer is made of a thermosetting resin or gel and is formed by thermosetting the resin or gel while the individual steps on the configurable foot orthosis.

13. The configurable foot orthosis of claim 1, further comprising a cover layer, being the topmost layer, wherein the cover layer protects the configurable foot orthosis from wear and tear.

14. The configurable foot orthosis of claim 13, wherein the cover layer is made of a material selected from the group consisting of leather, hypoallergenic expanded thermoplastic and polyurethane foam.

15. (canceled)

16. (canceled)

17. The configurable foot orthosis of claim 1, wherein the footwear is selected from a group consisting of a skate, a ski boot, a running shoe, a hiking shoe, a hiking boot and a workboot.

18. A postural adjustment system, comprising:
a configurable foot orthosis according to claim 1;
an imaging unit; and
a processing unit operatively connected to the configurable foot orthosis and the imaging unit, the processing unit being so configured so as to:
acquire at least one digital image of an individual from the imaging unit;
process the at least one digital image;
identify one or more postural deviations of the individual;
generate adjustments variables for the plurality of position adjusters in accordance with the one or more postural deviations;
providing commands to adjust the plurality of position adjusters in accordance with the adjustment variables in order to correct the one or more postural deviations of the individual.

19. The postural adjustment system of claim 18, further comprising a correspondence table and wherein the adjustments to be made to the plurality of position adjusters a generated by applying the correspondence table to the one or more postural deviations.

20. The postural adjustment system of claim 18, further comprising a configuration interface for applying the adjustment commands provided by the processing unit to the plurality of position adjusters of the configurable back orthosis.

21. The postural adjustment system of claim 18, further comprising a plurality of markers adapted to be placed at specific anatomical locations on the body of the individual and a biomechanical database, the processing unit being further so configured so as to:
obtain 3D coordinates of the markers from the at least one digital image;
compare the obtained 3D coordinates with corresponding expected 3D coordinates in a healthy individual from the biomechanical database; and
compute biomechanical parameters from the 3D coordinates comparison;
wherein the one or more postural deviations are identified using the computed biomechanical parameters and the biomechanical database.

22. The postural adjustment system of claim 18, further comprising a pressure point matrix for fine tuning the adjustments variables before providing commands to adjust the plurality of position adjusters, the pressure point matrix comprising:
a body supporting a plurality of gauges, each gauge having an associated actuator and an associated sensor, the body including a top surface having a plurality of perforations therethrough, each perforation being configured so as to allow a corresponding gauge to protrude from the body;
wherein the fine tuning is performed by having the individual step onto the pressure point matrix and adjusting the adjustments variables until the processing unit determines that there are no postural deviations remaining.

23. (canceled)

24. (canceled)
